SEQUENCE LISTING (41 sequences)

SEQ ID NO: 1 - DNA, 41 bp, Homo sapiens
caggactgggcctcctgcgtrccaggtggggctggtggggt

SEQ ID NO: 2 - DNA, 52 bp, Homo sapiens
ccagaaaagagggtacttcacaccagstctgtcactggagatggtcagagac

SEQ ID NO: 3 - DNA, 52 bp, Homo sapiens
actcttctatggcctactttctactgstatttgtgttactcatgctacccat

SEQ ID NO: 4 - DNA, 52 bp, Homo sapiens
agaagtttggaatggcaagaagtaagrcctgctagctaggcagggactggat

SEQ ID NO: 5 - DNA, 52 bp, Homo sapiens
aaattggacaggagactgaggagaaaygccgggagaggcaacaaccgccctc

SEQ ID NO: 6 - DNA, 25 bp, Artificial Sequence
  Forward primer for rs1048201
atgatatacatatctgacttcccaa

SEQ ID NO: 7 - DNA, 27 bp, Artificial Sequence
  Reverse primer for rs1048201
aagagactggtataaaatcagaattca

SEQ ID NO: 8 - DNA, 45 bp, Artificial Sequence
  Genotyping primer for rs1048201
cgtgccgctcgtgatagaatagctccaggatttgtgtgctgttgc

SEQ ID NO: 9 - DNA, 26 bp, Artificial Sequence
  Forward primer for rs6534367
ttcttctattatgcarttgtttgaag

SEQ ID NO: 10 - DNA, 28 bp, Artificial Sequence
  Reverse primer for rs6534367
ttacattctcaactagtgttctacattg

SEQ ID NO: 11 - DNA, 45 bp, Artificial Sequence
  Genotyping primer for rs6534367
acgcacgtccacggtgatttataaatrtatacaattttgattatt

SEQ ID NO: 12 - DNA, 18 bp, Artificial Sequence
  Forward primer for rs308428
gcatgttttgggaaccaa

SEQ ID NO: 13 - DNA, 22 bp, Artificial Sequence
  Reverse primer for rs308428
attaaaaccctccattgactcc

SEQ ID NO: 14 - DNA, 45 bp, Artificial Sequence
  Genotyping primer for rs308428
cgtgccgctcgtgatagaatgaagtttggaatggcaagaagtaag

SEQ ID NO: 15 - DNA, 19 bp, Artificial Sequence
  Forward primer for rs3741208
acaggtaaagcttccttcc

SEQ ID NO: 16 - DNA, 17 bp, Artificial Sequence
  Reverse primer for rs3741208
gccatcaggaggagaga

SEQ ID NO: 17 - DNA, 45 bp, Artificial Sequence
  Genotyping primer for rs3741208
cgactgtaggtgcgtaactcgagggcvgttgttgcctctcccggy

SEQ ID NO: 18 - DNA, 18 bp, Artificial Sequence
  Forward primer for rs2585
aatgtcacctgtgcctgc

SEQ ID NO: 19 - DNA, 22 bp, Artificial Sequence
  Reverse primer for rs2585
ttaaagacaaaacccaagcatg

SEQ ID NO: 20 - DNA, 25 bp, Artificial Sequence
  Genotyping primer for rs2585
tgcggcccgtgtttgactyaactca

SEQ ID NO: 21 - DNA, 18 bp, Artificial Sequence
  Forward primer for DL1002505
tgtccggcagcaggagga

SEQ ID NO: 22 - DNA, 18 bp, Artificial Sequence
  Reverse primer for DL1002505
acgcacactgccagacca

SEQ ID NO: 23 - DNA, 25 bp, Artificial Sequence
  Genotyping primer for DL1002505
aggactgggcctcctgcgtgctggc

SEQ ID NO: 24 - DNA, 18 bp, Artificial Sequence
  Forward primer for rs308395
gaggcacgtccatacttg

SEQ ID NO: 25 - DNA, 18 bp, Artificial Sequence
  Reverse primer for rs308395
cagcgtctcacacactga

SEQ ID NO: 26 - DNA, 25 bp, Artificial Sequence
  Genotyping primer for rs308395
ctcttctatggcctactttctactg

SEQ ID NO: 27 - DNA, 18 bp, Artificial Sequence
  Forward primer for rs11938826
ttggggaaggctgataat

SEQ ID NO: 28 - DNA, 18 bp, Artificial Sequence
  Reverse primer for rs11938826
gccatatttcggctaaca

SEQ ID NO: 29 - DNA, 27 bp, Artificial Sequence
  Genotyping primer for rs11938826
cccagaaaagagggtacttcacaccag

SEQ ID NO: 30 - DNA, 18 bp, Artificial Sequence
  Forward primer for rs3213221
taggacggaggccaggtc

SEQ ID NO: 31 - DNA, 17 bp, Artificial Sequence
  Reverse primer for rs3213221
aggtgcccctcccaaac

SEQ ID NO: 32 - DNA, 25 bp, Artificial Sequence
  Genotyping primer for rs3213221
aattttacacgagggktgaccatct

SEQ ID NO: 33 - DNA, 36 bp, Artificial Sequence
  Genotyping primer for rs308428
gggaaccaaaagaagtttggaatggcaagaagtaag

SEQ ID NO: 34 - DNA, 22 bp, Artificial Sequence
  Forward primer for rs308395
atgtctgaaatgtcacagcact

SEQ ID NO: 35 - DNA, 23 bp, Artificial Sequence
  Reverse primer for rs308395
aacacatawtgttgagtgtgtgg

SEQ ID NO: 36 - DNA, 42 bp, Artificial Sequence
  Genotyping primer for rs308395
cacagcacttagtcttactcttctatggcctactttctactg

SEQ ID NO: 37 - DNA, 26 bp, Artificial Sequence
  Forward primer for rs11938826
tatggccaatagaaaagagtataatc

SEQ ID NO: 38 - DNA, 24 bp, Artificial Sequence
  Reverse primer for rs11938826
atcaaagggttgttaaacagtctc

SEQ ID NO: 39 - DNA, 28 bp, Artificial Sequence
  Genotyping primer for rs11938826
tcccagaaaagagggtacttcacaccag

SEQ ID NO: 40 - DNA, 23 bp, Artificial Sequence
  Reverse primer for rs308395
aacacataatgttgagtgtgtgg

SEQ ID NO: 41 - DNA, 23 bp, Artificial Sequence
  Reverse primer for rs308395
aacacatattgttgagtgtgtgg

(19) United States
(12) Patent Application Publication
(10) Pub. No.: US 2013/0017975 A1
(43) Pub. Date: Jan. 17, 2013

(54) SINGLE NUCLEOTIDE POLYMORPHISM FOR PREDICTING PROGNOSIS OF HEPATOCELLULAR CARCINOMA

(71) Applicants: Young Hwa Chung, Seoul (KR); Neung Hwa Park, Ulsan (KR); Eun Sil Yu, Seoul (KR); Young-Joo Lee, Seoul (KR); Jeong A Kim, Seoul (KR); Dan-Bi Lee, Seoul (KR); Sae-Hwan Lee, Cheonan-shi (KR); Jong-Eun Lee, Seoul (KR)

(72) Inventors: Young Hwa Chung, Seoul (KR); Neung Hwa Park, Ulsan (KR); Eun Sil Yu, Seoul (KR); Young-Joo Lee, Seoul (KR); Jeong A Kim, Seoul (KR); Dan-Bi Lee, Seoul (KR); Sae-Hwan Lee, Cheonan-shi (KR); Jong-Eun Lee, Seoul (KR)

(73) Assignee: University of Ulsan Foundation For Industry Cooperation, Ulsan (KR)

(21) Appl. No.: 13/636,782

(22) PCT Filed: Mar. 22, 2011
(86) PCT No.: PCT/KR2011/001964

§ 371 Date: Sep. 24, 2012

(30) Foreign Application Priority Data
Mar. 22, 2010 (KR) 10-2010-0025371
Mar. 22, 2011 (KR) 10-2011-0025174

(51) Int. Cl. C40B 30/04; C40B 40/06; C12Q 1/68; C07H 21/04

(52) U.S. Cl. 506/9; 536/23.5; 506/16; 435/6.11

(57) ABSTRACT

Single nucleotide polymorphisms (SNP) for predicting prognosis of hepatocellular carcinoma after curative surgical resection are provided. The SNPs have a significant correlation with an over-expression of MTA1 which is useful prognostic factor for prediction of prognosis or poor survival after curative surgical resection of hepatocellular carcinoma. Therefore, the SNPs can be used in developing micro-arrays or test kits for prediction of the prognosis of hepatocellular carcinoma, and in screening drugs to improve poor prognosis of hepatocellular carcinoma after curative surgical resection.

### TECHNICAL FIELD

The present invention relates to a single nucleotide polymorphism (SNP) for predicting prognosis of hepatocellular carcinoma, in which the SNP shows a significant correlation with an over-expression of metastatic tumor antigen 1 (MTA1) which is useful prognostic factor for predicting recurrence or poor survival after hepatocellular carcinoma surgery, a micro-array or a test kit for predicting prognosis of hepatocellular carcinoma using the same, and a method for screening a drug to improve prognosis of hepatocellular carcinoma.

### BACKGROUND ART

Hepatocellular carcinoma (HCC) is the most common and serious cancer to be in third place among all of malignant tumors from the viewpoint of cancer development and deaths in this country. The hepatocellular carcinoma is one of the most hypervascular tumors.

The most effective modality to treat HCC is surgical resection or liver transplantation. However, only 10% to 20% of the patients with hepatocellular carcinoma can be operated due to the size and number of the tumor that cannot be removed, a bad liver function, all kinds of intrahepatic or extrahepatic metastases, and the like. Even with regard to the operable patients with hepatocellular carcinoma, frequent postoperative recurrence is a main limiting factor in survival for a long time period.

The development and rapid progression of hepatocellular carcinoma involve various mechanisms. Among them, the promotion of angiogenesis by hypoxia plays very important roles. In addition, under the hypoxic environment in liver, metastatic tumor antigen 1 (MTA1) contributes to angiogenesis of malignant tumor by enhancing the expression of a vascular endothelial growth factor (VEGF) by structurally stabilizing hypoxia inducible factor 1 (HIF 1) (Moon E J, et al., 2004; Moon E J, et al., 2006).

The inventors found that MTA1 is closely associated with a tumor size, a macroscopic shape of tumor, a histological differentiation of tumor, and the invasion to the surrounding tissues and micro-vessel of tumor from the research that was performed by targeting total 506 patients after curative surgical resection in order to confirm the correlation between the above described MTA1 expression and clinical properties of hepatocellular carcinoma. In addition, it was found that as MTA1 is strongly expressed, the relapse rate is high and the survival rate is low after curative surgical resection.

In conclusion, MTA1 is considered to play very important roles in the processes of development, progression, relapses in liver and distant metastases of hepatocellular carcinoma and also it was found that the MTA1 is a useful prognosis factor that can predict recurrence and survivals of patients suffered from hepatocellular carcinoma after curative surgical resection (Ryu S H, et al., 2007).

Meanwhile, after infection of hepatitis B virus, about 5% to 10% becomes chronic hepatitis B, and some of them may progress to cirrhosis, or rarely hepatocellular carcinoma. Like this, it is understood that various clinical progressions exhibited after the infection of hepatitis B virus depend on a difference of each individual's genetic predisposition as well as a difference of virus itself.

Genetic predisposition means that there is a tiny difference in various genes between individuals. Recently, the level of the difference has been reported from individual to individual through genome research. Among genetic variation, single nucleotide polymorphisms (SNPs) have been known to change the gene functions, and 710,000 polymorphisms in approximately 11 million SNPs have been reported in human genome so far (NCBI, dbSNP).

Recently, so many researches are being actively carried out to determine whether a tiny change in the base sequence actually influences susceptibilities to a certain disease, and to find the genetic variations predisposing the disease (Ludwig J A, and Weinstein J N, 2005; Suh Y, and Vijg J, 2005; Chanock S, 2001).

### DISCLOSURE

### Technical Problem

In order to address the above problems in the conventional technologies, the inventors were tried to identify a correlation between MTA1 expression in tissues of liver cancer and SNPs of important genes in angiogenesis, such as MTA1, VEGF, IGF2, HIF-1α, and FGF2, in which MTA1 is a protein relating to angiogenesis that is considered to be relevant to recurrence or survival rate of the hepatocellular carcinoma as well as the formation of tumor by the hepatocellular carcinoma. In addition, the inventors analyzed the effects of SNPs of angiogenesis gene on prognosis such as the recurrence or survival rate after curative surgical resection of the patients suffered from the hepatocellular carcinoma. Accordingly, the present invention was completed.

Accordingly, an object of the present invention is to provide single nucleotide polymorphisms (SNP) exhibiting a significant correlation with an over-expression of MTA1 that is a useful prognosis factor for predicting recurrence and poor survival after the operation of hepatocellular carcinoma.

In addition, another object of the present invention is to provide a micro-array for predicting prognosis of hepatocellular carcinoma using the single nucleotide polymorphisms (SNP).

In addition, still another object of the present invention is to provide a test kit for predicting prognosis of hepatocellular carcinoma.

In addition, still another object of the present invention is to provide a method for predicting prognosis of hepatocellular carcinoma using the sing nucleotide polymorphisms (SNP).

In addition, still another object of the present invention is to provide a method for screening a drug for improving prognosis of hepatocellular carcinoma using the single nucleotide polymorphism (SNP).

### Technical Solution

In order to achieve the above objects, an exemplary embodiment of the present invention provides a single nucleotide polymorphism (SNP) for predicting prognosis of hepatocellular carcinoma, in which the SNP includes at least one polynucleotide selected from the group consisting of GA or AA genotype in IVS4-81 (DL1002505) (SEQ ID NO. 1) of MTA1 gene; GG genotype in 24684C/G (rs11938826) [SEQ ID NO. 2], GG genotype in −989C/G (rs308395) [SEQ ID NO. 3], or GG genotype of 16578A/G (rs308428) [SEQ ID NO. 4] of FGF2 gene; and CC or CT genotype in −13021C/T (rs3741208) [SEQ ID NO. 5] of IGF2 gene; or a complementary nucleotide thereof.

In addition, an exemplary embodiment of the present invention provides a micro-array for predicting prognosis of hepatocellular carcinoma, in which the micro-array includes the polynucleotide of the single nucleotide polymorphism (SNP) for predicting prognosis of hepatocellular carcinoma, a polypeptide encoded by the same, or cDNA thereof.

In addition, an exemplary embodiment of the present invention provides a test kit for predicting prognosis of hepatocellular carcinoma, including the micro-array.

In addition, an embodiment of the present invention provides a test kit for predicting prognosis of hepatocellular carcinoma, using a single-base extension (SBE) reaction in order to genotyping SNP.

The test kit for predicting prognosis of hepatocellular carcinoma using the single-base extension reaction is designed to confirm whether GA or AA genotype in IVS4-81 (DL1002505) [SEQ ID NO. 1] of MTA1 gene; and GG genotype in 24684C/G (rs11938826) [SEQ ID NO. 2], GG genotype in −989C/G (rs308395) [SEQ ID NO. 3], or GG genotype in 16578A/G (rs308428) [SEQ ID NO. 4] of FGF2 gene exist.

An embodiment of the present invention provides a test kit for predicting prognosis of hepatocellular carcinoma, using a single-base extension reaction, including a forward primer for amplifying 16578A/G (rs308428) region of FGF2 gene; a reverse primer for amplifying 16578A/G (rs308428) region of FGF2 gene; a primer for genotyping 16578A/G (rs308428) region of FGF2 gene; a forward primer for amplifying IVS4-81 (DL1002505) region of MTA1 gene; a reverse primer for amplifying IVS4-81 (DL1002505) region of MTA1 gene; a primer for genotyping IVS4-81 (DL1002505) region of MTA1 gene; a forward primer for amplifying −989C/G (rs308395) region of FGF2 gene; a reverse primer for amplifying −989C/G (rs308395) region of FGF2 gene; a primer for genotyping −989C/G (rs308395) region of FGF2 gene; a forward primer for amplifying 24684C/G (rs11938826) region of FGF2 gene; a reverse primer for amplifying 24684C/G (rs11938826) region of FGF2 gene; and a primer for genotyping 24684C/G (rs11938826) region of FGF2 gene.

According an embodiment, in the test kit for predicting prognosis of hepatocellular carcinoma, the forward primer for amplifying 16578A/G (rs308428) region of FGF2 gene may be a primer of SEQ ID NO. 12; the reverse primer for amplifying 16578A/G (rs308428) region of FGF2 gene may be a primer of SEQ ID NO. 13; the primer for genotyping 16578A/G (rs308428) region of FGF2 gene may be a primer of SEQ ID NO. 33; the forward primer for amplifying IVS4-81 (DL1002505) region of MTA1 gene may be a primer of SEQ ID NO. 21; the reverse primer for amplifying IVS4-81 (DL1002505) region of MTA1 gene may be a primer of SEQ ID NO. 22; the primer for genotyping IVS4-81 (DL1002505) region of MTA1 gene may be a primer of SEQ ID NO. 23; the forward primer for amplifying −989C/G (rs308395) region of FGF2 gene may be a primer of SEQ ID NO. 34; the reverse primer for amplifying −989C/G (rs308395) region of FGF2 gene may be a primer of SEQ ID NO. 35; the primer for genotyping −989C/G (rs308395) region of FGF2 gene may be a primer of SEQ ID NO. 36; the forward primer for amplifying 24684C/G (rs11938826) region of FGF2 gene may be a primer of SEQ ID NO. 37; the reverse primer for amplifying 24684C/G (rs11938826) region of FGF2 gene may be a primer of SEQ ID NO. 38; and the primer for genotyping 24684C/G (rs11938826) region of FGF2 gene may be a primer of SEQ ID NO. 39.

In addition, the present invention provides a method for predicting prognosis of hepatocellular carcinoma, the method including a step of obtaining a nucleic acid sample from a clinical specimen; and a step of determining a nucleotide sequence of at least any one polymorphism regions of polynucleotide selected from the group consisting of GA or AA genotype in IVS4-81 (DL1002505) of MTA1 gene; GG genotype in 24684C/G (rs11938826), GG genotype in −989C/G (rs308395), or GG genotype in 16578A/G (rs308428) of FGF2 gene; and CC or CT genotype in −13021C/T (rs3741208) of IGF2 gene, or a complementary nucleotide thereof.

The step of determining the nucleotide sequence of the polymorphism region may include a step of hybridizing the nucleic acid sample to the micro-array fixed with the polynucleotide or the complementary nucleotide thereof and a step of detecting a hybridization result thus obtained.

In addition, the present invention provides a method for screening a drug for improving prognosis of hepatocellular carcinoma, the method including a step of contacting a polypeptide encoded by the polynucleotide or the complementary nucleotide thereof of the single nucleotide polymorphism (SNP) for predicting prognosis of hepatocellular carcinoma with a candidate material; and a step of determining whether the candidate material has activity to enhance or inhibit a function of the polypeptide.

According to the present invention, a micro-array or a test kit for predicting prognosis of hepatocellular carcinoma can be developed by using a SNP by providing a single nucleotide polymorphism (SNP) exhibiting a significant correlation with an over-expression of MTA1 that is a useful prognosis factor for predicting recurrence or a poor survival after the operation of hepatocellular carcinoma, and a poor prognosis after the operation of hepatocellular carcinoma can be improved by screening a drug for improving prognosis of hepatocellular carcinoma.

### DESCRIPTION OF DRAWINGS

FIG. 1 shows a cumulative recurrence rate of hepatocellular carcinoma according to an expression level of MTA1;

FIG. 2 shows a cumulative survival rate of hepatocellular carcinoma according to an expression level of MTA1; and

FIGS. 3 to 5 show results of genotyping using a test kit for predicting prognosis of hepatocellular carcinoma according to an embodiment of the present invention.

In order to achieve the above objects, the present invention provides single nucleotide polymorphisms (SNP) for predicting prognosis of hepatocellular carcinoma, the SNP including at least one polynucleotide selected from the group consisting of GA or AA genotype in IVS4-81(DL1002505) [SEQ ID NO. 1] of MTA1 gene; GG genotype in 24684C/G (rs11938826) [SEQ ID NO. 2], GG genotype in −989C/G (rs308395) [SEQ ID NO. 3], or GG genotype in 16578A/G(rs308428) [SEQ ID NO. 4] of FGF2 gene; and CC or CT genotype in −13021C/T (rs3741208) [SEQ ID NO. 5] in IGF2 gene, or a complementary nucleotide thereof.

The single nucleotide polymorphism (SNP) exhibits a significant correlation with an over-expression of a metastatic tumor antigen 1 (MTA1).

In addition, the prognosis of hepatocellular carcinoma may be related to prognosis in patients with hepatocellular carcinoma treated with curative surgical resection, and may be related to any one marker selected from a rate of tumorigenesis, a rate of recurrence risk, or a survival rate in hepatocellular carcinoma.

In addition, the present invention provides a micro-array for predicting prognosis of hepatocellular carcinoma, the micro-array including the polynucleotide of the single nucleotide polymorphism (SNP) for predicting the prognosis of hepatocellular carcinoma, a polypeptide encoded by the same, or cDNA thereof.

The micro-array for predicting the prognosis of hepatocellular carcinoma may be manufactured by the general method known by a person of ordinary skill in the art, and for example, the polynucleotide that is included in the micro-array for predicting the prognosis of hepatocellular carcinoma may be fixed to a substrate coated with an active group selected from the group consisting of an amino-silane, a poly-L-lysine, and aldehyde, and the substrate may be selected from the group consisting of a silicon wafer, glass, quartz, metal, and plastic. The method for fixing the polynucleotide to the substrate may include a micropipetting method using a piezoelectric way, a method suing a spotter of a pin type, and the like.

In addition, the present invention provides a test kit for predicting prognosis of hepatocellular carcinoma, the test kit including the micro-array.

The test kit according to the present invention may further include a set of primers that is used for isolating and amplifying DNA including a relevant SNP from a clinical specimen in addition to the micro-array of the present invention. The appropriate set of primers may be easily designed by a person of ordinary skill in the art with reference to the sequences of the present invention.

In addition, an embodiment of the present invention provides a test kit for predicting prognosis of hepatocellular carcinoma using a single-base extension (SBE) reaction in order for genotyping SNP. In this case, the primers for an amplification (Forward direction and Reverse direction) and extension (Genotyping) should be designed for the single-base extension (SBE).

The test kit for predicting prognosis of hepatocellular carcinoma using the single-base extension reaction may be a test kit for analyzing a genotype of SNaPshot method.

The test kit for predicting prognosis of hepatocellular carcinoma using the single-base extension reaction is designed in order to confirm whether GA or AA genotype in IVS4-81 (DL1002505) [SEQ ID NO. 1] of MTA1 gene; and GG genotype in 24684C/G (rs11938826) [SEQ ID NO. 2], GG genotype in −989C/G (rs308395) [SEQ ID NO. 3], or GG genotype in 16578A/G (rs308428) [SEQ ID NO. 4] of FGF2 gene exist.

An embodiment of the present invention provides a test kit for predicting prognosis of hepatocellular carcinoma, the test kit including a forward primer for amplifying 16578A/G (rs308428) region of FGF2 gene; a reverse primer for amplifying 16578A/G (rs308428) region of FGF2 gene; a primer for genotyping 16578A/G (rs308428) region of FGF2 gene; a forward primer for amplifying IVS4-81 (DL1002505) region of MTA1 gene; a reverse primer for amplifying IVS4-81 (DL1002505) region of MTA1 gene; a primer for genotyping IVS4-81 (DL1002505) region of MTA1 gene; a forward primer for amplifying −989C/G (rs308395) region of FGF2 gene; a reverse primer for amplifying −989C/G (rs308395) region of FGF2 gene; a primer for genotyping −989C/G (rs308395) region of FGF2 gene; a forward primer for amplifying 24684C/G (rs11938826) region of FGF2 gene; a reverse primer for amplifying 24684C/G (rs11938826) region of FGF2 gene; and a primer for genotyping 24684C/G (rs11938826) region of FGF2 gene, and using a single-base extension reaction.

According to an embodiment, in the test kit for predicting the prognosis of hepatocellular carcinoma, the forward primer for amplifying 16578A/G (rs308428) region of FGF2 gene may be a primer of SEQ ID NO. 12; the reverse primer for amplifying 16578A/G (rs308428) region of FGF2 gene may be a primer of SEQ ID NO. 13; the primer for genotyping 16578A/G (rs308428) region of FGF2 gene may be a primer of SEQ ID NO. 33; the forward primer for amplifying IVS4-81 (DL1002505) region of MTA1 gene may be a primer of SEQ ID NO. 21; the reverse primer for amplifying IVS4-81 (DL1002505) region of MTA1 gene may be a primer of SEQ ID NO. 22; the primer for genotyping IVS4-81 (DL1002505) region of MTA1 gene may be a primer of SEQ ID NO. 23; the forward primer for amplifying −989C/G (rs308395) region of FGF2 gene may be a primer of SEQ ID NO. 34; the reverse primer for amplifying −989C/G (rs308395) region of FGF2 gene may be a primer of SEQ ID NO. 35; the primer for genotyping −989C/G (rs308395) region of FGF2 gene may be a primer of SEQ ID NO. 36; the forward primer for amplifying 24684C/G (rs11938826) region of FGF2 gene may be a primer of SEQ ID NO. 37; the reverse primer for amplifying 24684C/G (rs11938826) region of FGF2 gene may be a primer of SEQ ID NO. 38; and the primer for genotyping 24684C/G (rs11938826) region of FGF2 gene may be a primer of SEQ ID NO. 39.

In addition, the present invention provides a method for predicting prognosis of hepatocellular carcinoma, the method including a step of obtaining a nucleic acid sample from a clinical specimen; and a step of determining a nucleotide sequence of at least any one polymorphism region of a polynucleotide selected from the group consisting of GA or AA genotype in IVS4-81 (DL1002505) of MTA1 gene; GG genotype in 24684C/G (rs11938826), GG genotype in −989C/G (rs308395), or GG genotype in 16578A/G (rs308428) of FGF2 gene; and CC or CT genotype in −13021C/T (rs3741208) of IGF2 gene, or a complementary nucleotide thereof. The nucleic acid may include DNA, mRNA, or cDNA synthesized from mRNA.

The step of determining the nucleotide sequence of the polymorphism region may include a step of hybridizing the nucleic acid sample to the micro-arrays fixed with the polynucleotide or the complementary nucleotide thereof, and a step of detecting the hybridization result thus obtained.

For example, DNA is isolated from a tissue, body fluid, or cell of objects; amplified through PCR; and then SNP is analyzed. The SNP analysis may be performed by using the known general method. For example, the SNP analysis may be performed by using a real time PCR system or by directly determining the nucleotide sequence of nucleic acid by a dideoxy method. Alternatively, the SNP analysis may be performed by determining the nucleotide sequence of polymorphism region by measuring the degree of hybridization obtained by hybridizing the DNA with a probe including the sequence of SNP region or a complementary probe thereof, or may be performed by using allele-specific probe hybridization, allele-specific amplification, sequencing, 5′ nuclease digestion, molecular beacon assay, oligonucleotide ligation assay, size analysis, single-stranded conformation polymorphism, and the like.

In addition, the present invention provides a method for screening a drug for improving prognosis of hepatocellular carcinoma, the method including a step of contacting a polypeptide encoded by the polynucleotide of a single nucleotide polymorphism (SNP) for predicting prognosis of hepatocellular carcinoma, or the complementary nucleotide thereof with a candidate material; and a step of determining whether the candidate material has activity to improve or inhibit a function of the polypeptide.

In the screening method of the present invention, the reaction between the polypeptide and the candidate material may be determined by using general methods used for determining whether the reaction between protein-protein and the reaction between protein-compound are occurred or not. For example, there may be a method for measuring activity after reacting the protein and the candidate material, a yeast two-hybrid, a search of phage-displayed peptide clone bonded to the protein, a high throughput screening (HTS) using a natural substance, chemical library, and the like, a drug hit HTS, a cell-based screening, a method for screening using DNA array, or the like.

In the screening method of the present invention, the candidate material may be individual nucleic acids, proteins, other extracts, natural substances, compounds, or the like, that are assumed to have potential to be a diagnostic agent for prognosis of hepatocellular carcinoma or are randomly selected according to a general selection method.

### BEST MODE

Hereinafter, the present invention will be described in more detail with reference to the following Examples. However, the present invention is not limited to the following Examples.

### EXAMPLE 1

### SNP Selection

A biallelic SNPs included in ±2 kb of angiogenesis-related genes, that is, VEGF, HIF1a, IGF2, FGF2, or MTA1, was subjected. As the positions of SNPs of the genes, 5′-nontranslation, promoter, exon, and gene loci regions were selected with reference to gene information (http://www.ncbi.nlm.nih.gov/project/SNP) that is already known.

The IDs of subjected SNPs are as follows:

rs699947, rs25648, rs3025000, rs3025010, rs3025035, rs3025040, rs10434, rs998584, rs45533131/rs1957757, rs2301113, rs2057482/rs2585, rs3802971, rs3213221, rs3741212, rs2239681, rs3741208, rs1004446, rs7924316, rs3842748, rs2070762/rs308395, rs308428, rs11938826, rs17472986, rs308442, rs308379, rs308381, rs6534367, rs1048201, rs3747676/rs4983413, and DL1002505.

Among the above SNPs, SNPs having a haplotype frequency of equal to or greater than 5% were selected, and the haplotype frequencies were analyzed by using PHASE software v2.1. In addition, linkage disequilibrium was analyzed by using Haploview program v3.2 (http://www.broad.mit.edu/mpg/haploview/index.php).

### EXAMPLE 2

### SNP Genotype Analysis

1. SNP Genotype Analysis

A primer set that can amplify the region including SNP of Example 1 and TaqMan probe including SNP region were manufactured by using primer express software. As the TaqMan probe, each of the probes that are suitable for wide type and mutant alleles was manufactured according to the sequence of SNP.

A probe was manufactured by tacking a fluorescent dye on one side of the TaqMan probe and tacking a quencher that can inhibit the color of the fluorescent dye on the other side. In this case, separate fluorescent dyes having different colors were tacked to the wild type and mutant alleles, respectively.

Three types of primers disclosed in the following Table 1 were mixed together, and then PCR reaction was performed by using the mixed primers to distinguish the mismatch of a pair of single nucleotide according to active property of exonuclesase of Taq polymerase.

TABLE 1

| rs No. | Strand | Primer Sequence | |
|---|---|---|---|
| rs1048201 | Forward | Forward | ATGATATACATATCTGACTTCCCAA |
| | | | (SEQ ID NO. 6) |
| | | Reverse | AAGAGACTGGTATAAAATCAGAATT |
| | | | CA (SEQ ID NO. 7) |
| | | Genotyping | CGTGCCGCTCGTGATAGAATAGCTCC |
| | | | AGGATTTGTGTGCTGTTGC |
| | | | (SEQ ID NO. 8) |
| rs6534367 | Forward | Forward | TTCTTCTATTATGCARTTGTTTGAAG |
| | | | (SEQ ID NO. 9) |
| | | Reverse | TTACATTCTCAACTAGTGTTCTACATT |
| | | | G (SEQ ID NO. 10) |
| | | Genotyping | ACGCACGTCCACGGTGATTTATAAAT |
| | | | RTATACAATTTTGATTATT |
| | | | (SEQ ID NO. 11) |
| rs308428 | Forward | Forward | GCATGTTTTGGGAACCAA |
| | | | (SEQ ID NO. 12) |
| | | Reverse | ATTAAAACCCTCCATTGACTCC |
| | | | (SEQ ID NO. 13) |
| | | Genotyping | CGTGCCGCTCGTGATAGAATGAAGTT |
| | | | TGGAATGGCAAGAAGTAAG |
| | | | (SEQ ID NO. 14) |
| rs3741208 | Reverse | Forward | acaggtaaagcttccttcc (SEQ ID NO. 15) |
| | | Reverse | gccatcaggaggagaga (SEQ ID NO. 16) |
| | | Genotyping | CgACTgTAggTgCgTAACTCgagggcVgagt |
| | | | tgcctctcccggY (SEQ ID NO. 17) |
| rs2585 | Forward | Forward | AATGTCACCTGTGCCTGC |
| | | | (SEQ ID NO. 18) |
| | | Reverse | TTAAAGACAAAACCCAAGCATG |
| | | | (SEQ ID NO. 19) |
| | | Genotyping | TGCGGCCCGTGTTTGACTYAACTCA |
| | | | (SEQ ID NO. 20) |
| DL1002505 | Forward | Forward | TGTCCGGCAGCAGGAGGA |
| | | | (SEQ ID NO. 21) |
| | | Reverse | ACGCACACTGCCAGACCA |
| | | | (SEQ ID NO. 22) |
| | | Genotyping | aggactgggcctcctgcgtgctggc(SEQ ID ND. 23) |
| rs308395 | Forward | Forward | gaggcacgtccatacttg (SEQ ID NO. 24) |
| | | Reverse | cagcgtctcacacactga (SEQ ID NO. 25) |
| | | Genotyping | ctcttctatggcctactttctactg (SEQ ID NO. 26) |
| rs11938826 | Forward | Forward | TTGGGGAAGGCTGATAAT |
| | | | (SEQ ID NO. 27) |
| | | Reverse | GCCATATTTCGGCTAACA |
| | | | (SEQ ID NO. 28) |
| | | Genotyping | CCCAGAAAAGAGGGTACTTCACACC |
| | | | AG (SEQ ID NO. 29) |
| rs3213221 | Reverse | Forward | taggacggaggccaggtc (SEQ ID NO. 30) |
| | | Reverse | aggtgcccctcccaaac (SEQ ID NO. 31) |
| | | Genotyping | aatatacacgagggKtgaccatct (SEQ ID NO. 32) |

A final SNP marker result was determined after verifying compatibility of the results that are read independently by more than two researchers. A result of individual SNP marker is represented by major allele homozygote, heterozygote, or minor allele homozygote according to a single nucleotide polymorphism allele. The results of the whole subjects were analyzed by the ratio of major and minor allele frequencies and frequencies of the three genotypes. The results were verified by Hardy-Weinberg equilibrium.

Student's t-test and chi-squared test were used and odds ratio was calculated to analyze the correlation between MTA1 expressions in hepatocellular carcinoma tissue and SNPs.

2. Result

GA or AA genotype in IVS4-81 (DL1002505) of MTA1 gene exhibited a significant correlation with positive for MTA1 (p=0.003). MTA1 positive rate was significantly high in the case of GG genotype (p=0.027) in 24684C/G (rs11938826), GG genotype (p=0.007) in −989C/G (rs308395), or GG genotype (p=0.007) in 16578A/G (rs308428) of FGF2 gene, respectively. CC or CT genotype in −13021C/T (rs3741208) in IGF2 gene exhibited a significant correlation with an over-expression of MTA1 (p=0.03). Meanwhile, other SNPs and haplotypes did not exhibit a significant correlation with an expression degree of MTA1.

### EXAMPLE 3

### Patient Characteristic

The experiment was performed for 506 patients suffered from recurrence of hepatocellular carcinoma in Asan Hospital from 1998 to 2003. Clinical characteristics of 506 patients are shown in the following Table 2. The follow-up survey of the patients was performed for an average period of 43 months (1 to 96 months) after curative surgical resection.

TABLE 2

| | Value of |
|---|---|
| Items | Characteristic |
| Age (Year, Mean ± SD) | 56 ± 10 |
| Sex (M:F) | 412:94 |
| Disease degree (CH:LC) | 138:368 |
| Evaluation of Liver Function (Child-Pugh class, A/B/C) | 383/73/50 |
| HCC Cause (HBV/HCV/Both/NBNC) | 397/29/8/72 |
| HCC Size (cm) | 4 (0.7-21) |
| Follow-up Survey Period After Hepatectomy (Month) | 43 (1-96) |

The recurrence of liver cancer and survival were determined by using a medical record at the last day of follow-up survey. In the case of the patients that were not followed up for 3 months, it was evaluated by visiting near residences of the patients.

### EXAMPLE 4

### MTA1 Immunohistochemical Staining Evaluation

1. MTA1 Immunohistochemical Staining in Human Hepatocellular Carcinoma Tissue

MTA1 immunochemical staining to hepatocellular carcinoma tissue was performed by using an avidin biotin peroxidase complex method and a color reaction was determined with 3,3′-diaminobenzidin and LSAB kit (DAKO, Carpentaria, Calif., USA).

A paraffin-embedded micro-dissected specimen of tissue harvested from hepatocellular carcinoma and a non-neoplastic region around hepatocellular carcinoma was treated with xylene to remove paraffin and re-hydrated with alcohols with gradual concentrations. The slice of liver tissue prepared was treated with 3% hydrogen peroxide for 10 minutes to block activity of endogenous peroxidase.

In order to amplify the reaction upon an immunochemical staining, antigen retrieval was performed by using a buffer solution of citric acid (pH 6.0) for 10 minutes in a steam oven. A primary antigen (Santa Cruz Biochemistry, Santa Cruz, Calif., USA) against MTA1 was diluted to be 1:200 to use and stained with a secondary biotinylated antibody and an avidin biotin complex reagent. A negative staining was performed with Harris hematoxylin.

A negative control was prepared as follows and then used: a tissue specimen was put in a Tris buffer physiological saline including 2% goat serum and 1% bovine serum albumin was used instead of a primary antibody. Among each of immunochemical staining slices, the region exhibiting the strongest staining reaction and also representing the whole tissue remarks most well was selected and then evaluated.

The intensity of MTA1 staining was determined as a rate of cell exhibiting an immune staining positive among the total cells and was defined as follows: 1) when the total cells were not stained entirely, it was defined as “none,” 2) when some of the cells exhibited a positive staining, but the rate was not greater than 50%, it was defined as “1+(+),” and 3) when greater than 50% of the total cells exhibited a positive staining in the immunochemical staining, it was defined as 2+(++).

At least two observers did not know the results each other, and evaluated the intensity of the staining. When the observers had different evaluations each other, a score was adjusted through the reevaluation.

In order to analysis a cumulative recurrence and cumulative survival rate, Kaplan-Meier method and log-rank test were used.

2. Result

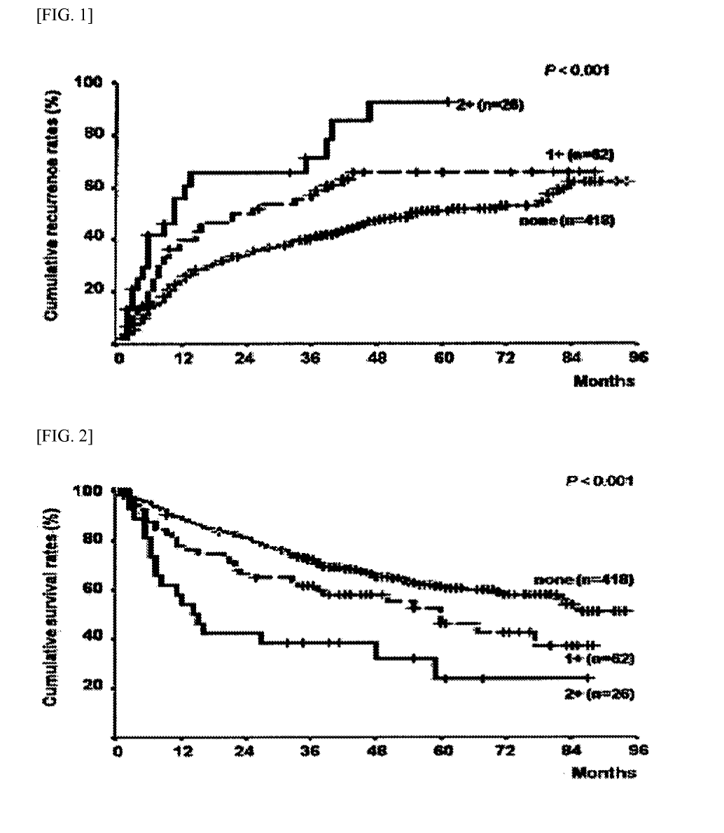

As illustrated in FIG. 1, the cumulative recurrences of MTA1-positive hepatocellular carcinoma at 1-year, 3-year, and 5-year were significantly higher than those of MTA1-negative hepatocellular carcinoma. The cumulative recurrences of the patient having high level of MTA1 expression (++) at 1-year and 3year were 41% and 71%, respectively, which are also significantly high level as compared with the patients having +level of MTA1 expression (39%, 54%) and the patients having negative of MTA1 expression (25%, 39%).

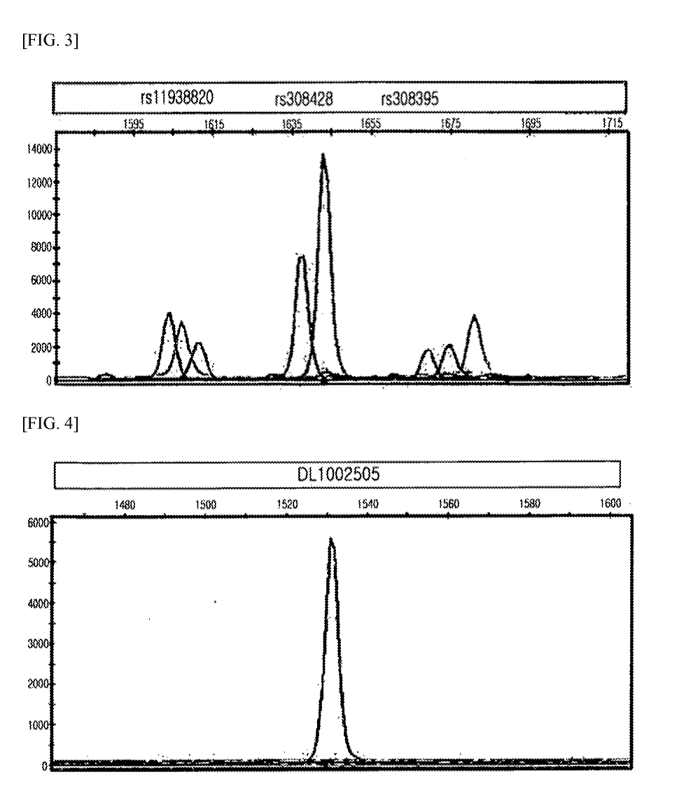

As illustrated in FIG. 2, the cumulative survival rates (54%, 39%) of the patients having MTA1-positive hepatocellular carcinoma at 1-year and 3-year were significantly shorter than those (89%, 72%) of the patients having MTA1-negative hepatocellular carcinoma.

### EXAMPLE 5

### Test Kit for Predicting Prognosis of Hepatocellular Carcinoma and Method of Analyzing Single Nucleotide Polymorphism for Predicting Prognosis of Hepatocellular Carcinoma

The primer sequences for an amplification (Forward direction and Reverse direction) and extension (Genotyping) in genotyping single nucleotide polymorphisms of rs308428, DL1002505, rs308395, and rs11938826, which are single nucleotide polymorphisms region exhibiting a significant correlation with MTA1-positive are shown in the following Table 3. The following SEQ ID NO. 35 (5′-AACACATAWTGTTGAGTGTGTGG-3′) may be a primer set including SEQ ID NO. 40 (5′-AACACATAATGTTGAGTGTGTGG-3′) and SEQ ID NO. 41 (5′-AACACATATTGTTGAGTGTGTGG-3′) in approximately 1:1.

TABLE 3

| rs No. | Strand | Primer Sequence | |
|---|---|---|---|
| rs308428 | Forward | Forward | 5′-GCATGTTTTGGGAACCAA-3(SEQ ID NO. 12) |
| | | Reverse | 5′-ATTAAAACCCTCCATTGACTCC-3′ |
| | | | (SEQ ID NO. 13) |
| | | Genotyping | 5′-GGGAACCAAAAGAAGTTTGGAATGGCAAGA |
| | | | AGTAAG-3′ (SEQ ID NO. 33) |
| DL1002505 | Forward | Forward | 5′-TGTCCGGCAGCAGGAGGA-3′(SEQ ID NO. 21) |
| | | Reverse | 5′-ACGCACACTGCCAGACCA-3′(SEQ ID NO. 22) |
| | | Genotyping | 5′-AGGACTGGGCCTCCTGCGTGCTGGC-3′ |
| | | | (SEQ ID NO. 23) |
| rs308395 | Forward | Forward | 5′-ATGTCTGAAATGTCACAGCACT-3′ |
| | | | (SEQ ID NO. 34) |
| | | Reverse | 5′-AACACATAWTGTTGAGTGTGTGG-3′ |
| | | | (SEQ ID NO. 35) |
| | | Genotyping | 5′-CACAGCACTTAGTCTTACTCTTCTATGGCCT |
| | | | ACTTTCTACTG-3′ (SEQ ID NO. 36) |
| rs11938826 | Forward | Forward | 5′-TATGGCCAATAGAAAAGAGTATAATC-3′ |
| | | | (SEQ ID NO. 37) |
| | | Reverse | 5′-ATCAAAGGGTTGTTAAACAGTCTC-3′ |
| | | | (SEQ ID NO. 38) |
| | | Genotyping | 5′-tcccagaaaagagggtacttcacaccag-3′ |
| | | | (SEQ ID NO. 39) |

1) PCR Amplification

First, a region including a single nucleotide polymorphism was amplified with Multiplex PCR. A composition of PCR reaction solution and condition for PCR reaction used for the PCR reaction are shown in the following Table 4 and Table 5. More specifically, DNA was isolated from a clinical sample and used as a DNA template for the PCR reaction. PCR reaction was performed by performing a predenaturation (1 cycle) at 95° C. for approximately 15 minutes; a denaturation at approximately 94° C. for approximately 30 seconds, annealing at approximately 55° C. for approximately 1 minutes and 30 seconds, and elongation at approximately 72° C. for approximately 1 minutes and 30 seconds as 1 cycle (35 cycles); and finally a final elongation at approximately 72° C. for approximately 10 minutes. The reactant was stored at approximately 4° C. In the above-described PCR reaction, the forward and reverse primers to each of single nucleotide polymorphisms in Table 3 described above were used as a primer for PCR reaction.

TABLE 4

| Reagent | Volume (Amount per 1 well) (μl) |
|---|---|
| 10X buffer | 1 |
| MgCl₂ (25 mM) | 1.4 |
| dNTP (10 mM) | 0.3 |
| primer pool (100 pmol/μl) | 0.24 |
| Taq (5 U/μl) | 0.2 |
| Distilled Water (DW) | 5.86 |
| DNA | 1 |
| Total | 10 |

TABLE 5

| Temperature | Time | Cycle |
|---|---|---|
| 95° C. | 15 minutes | 1 |
| 94° C. | 30 seconds | 35 |
| 55° C. | 1 minute and 30 seconds | |
| 72° C. | 1 minute and 30 seconds | |
| 72° C. | 10 minutes | 1 |
| 4° C. | ∞ | — |

2) PCR Product Purification: SAP & Exo I Treatment (10 μl)

In order to complete PCR reaction (SNaPshot reaction) for a primer extension, the PCR product was purified. That is, the following SAP & Exo I was treated to the PCR product. The composition of purified reaction materials and reaction condition of the product are shown in the following Table 6 and Table 7, respectively.

TABLE 6

| Material | Volume (μl) |
|---|---|
| SAP (1 unit/μl) | 5 |
| Exo I (10 unit/μl) | 0.2 |
| PCR Product | 4 |
| Distilled Water | 0.8 |
| Total | 10 |

TABLE 7

| Reaction Temperature | Reaction Time | |
|---|---|---|
| 37° C. | 1 | hour |
| 72° C. | 15 | minutes |

3) SNaPshot Reaction: PCR Reaction for One Base Extension

SNaPshot Reaction Premix and genotyping primer to each of single nucleotide polymorphisms in the above Table 3 were mixed with the purified PCR product, and then PCR reaction was performed. The composition of SNaPshot reaction materials and reaction condition are shown in the following Table 8 and Table 9, respectively. The PCR reaction was performed by performing a denaturation at approximately 96° C. for approximately 10 seconds, an annealing at approximately 50° C. for approximately 5 seconds, and an elongation at approximately 60° C. for approximately 30 seconds as 1 cycle (25 cycles).

TABLE 8

| Material | Volume (μl) |
|---|---|
| SNaPshot Ready Reaction Premix | 5 |
| Genotyping primer pool (0.15 pmol/μl) | 1 |
| Purified PCR Product | 2 |
| Distilled Water | 2 |
| Total | 10 |

TABLE 9

| Reaction Temperature | Reaction Time | |
|---|---|---|
| 96° C. | 10 | seconds |
| 50° C. | 5 | seconds |
| 60° C. | 30 | seconds |

| | |
|---|---|
| 25 cycle | — |

4) SAP Treatment: Process for Removing Non-reacted Oligonucleotide

In order to remove a non-reacted oligonucleotide, SAP was added and treated to the SNaPshot reaction product. The composition of the SAP treatment reaction materials and the reaction condition are shown in the following Table 10 and Table 11, respectively.

TABLE 10

| Material | Volume (μl) |
|---|---|
| SAP (1 unit/μl) | 1 |
| Distilled Water | 1 |

TABLE 11

| Reaction Temperature | Reaction Time | |
|---|---|---|
| 37° C. | 1 | hour |
| 72° C. | 15 | minutes |

5) Running

An analysis was performed by using an automatic sequencer, such as ABI 3730XL (Applied Biosystems, Calif., USA). At this time, the nucleotide sequence at a single nucleotide polymorphism (SNP) position was determined as a fluorescent color of analysis result.

6) Data Analysis

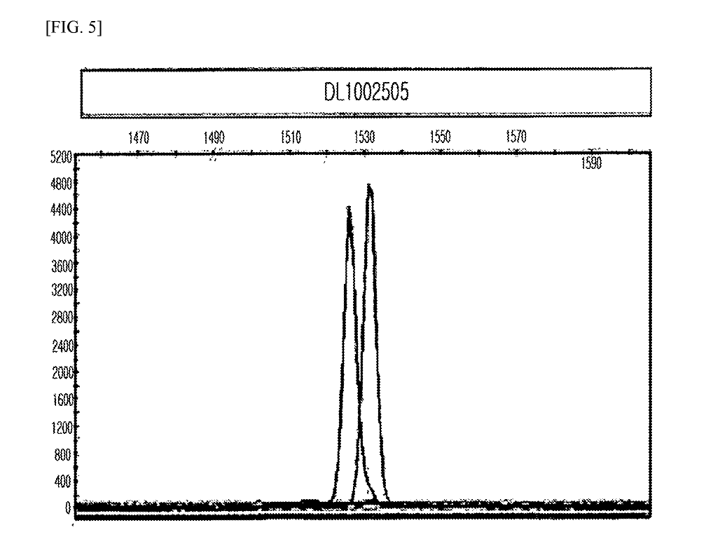

By analyzing the product subjected to the one base extension through ABI 3730XL that is an automatic sequencer, only the labeled part was detected to exhibit a peak, as illustrated in FIGS. 3 to 5. The sequence of the single nucleotide polymorphism could be confirmed by analyzing the fluorescent color exhibited as the peak (each of the bases used ddNTP labeled with the fluorescent dye with different colors each other).

When using a test kit for predicting prognosis of hepatocellular carcinoma and a method of analyzing a single nucleotide polymorphism for predicting prognosis of hepatocellular carcinoma according to an embodiment (Example 5) of the present invention, the single nucleotide polymorphisms of rs308428, rs308395, and rs11938826 could be multiplexly analyzed. However, DL1002505 that is a single nucleotide polymorphism exhibiting a significant correlation with prognosis of hepatocellular carcinoma could not be multiplexly analyzed, and was needed to be a separate analysis.

According to the present invention, a micro-array or a test kit for predicting prognosis of hepatocellular carcinoma can be developed by using a single nucleotide polymorphism (SNP) exhibiting a significant correlation with an over-expression of MTA1 that is a useful prognosis factor for predicting recurrence and a poor survival after curative surgical resection of hepatocellular carcinoma, and a poor prognosis of hepatocellular carcinoma can be improved by screening a drug for improving prognosis of hepatocellular carcinoma.

1. A single nucleotide polymorphism (SNP) for predicting prognosis of hepatocellular carcinoma, the SNP comprising at least one polynucleotide selected from the group consisting of GA or AA genotype in IVS4-81 (DL1002505) of MTA1 gene; GG genotype in 24684C/G (rs11938826), GG genotype in −989C/G (rs308395), or GG genotype in 16578A/G (rs308428) of FGF2 gene; and CC or CT genotype in −13021C/T (rs3741208) of IGF2 gene, or a complementary nucleotide thereof,

2. The single nucleotide polymorphism (SNP) for predicting the prognosis of hepatocellular carcinoma according to claim 1, wherein the single nucleotide polymorphism (SNP) has a significant correlation with an over-expression of metastatic tumor antigen 1 (MTA1).

3. The single nucleotide polymorphism (SNP) for predicting the prognosis of hepatocellular carcinoma according to claim 1, wherein the prognosis of hepatocellular carcinoma relates to prognosis of patients treated with curative surgical resection of hepatocellular carcinoma, and is evaluated by using any one marker selected from a rate of tumorigenesis, a rate of recurrence risk, or a survival rate in hepatocellular carcinoma.

4. A test kit for predicting prognosis of hepatocellular carcinoma, the test kit using a single-base extension reaction and comprising:
a forward primer for amplifying 16578A/G (rs308428) region of FGF2 gene;
a reverse primer for amplifying 16578A/G (rs308428) region of FGF2 gene;
a primer for genotyping 16578A/G (rs308428) region of FGF2 gene;
a forward primer for amplifying IVS4-81 (DL1002505) region of MTA1 gene;
a reverse primer for amplifying IVS4-81 (DL1002505) region of MTA1 gene;
a primer for genotyping IVS4-81 (DL1002505) region of MTA1 gene;
a forward primer for amplifying −989C/G (rs308395) region of FGF2 gene;
a reverse primer for amplifying −989C/G (rs308395) region of FGF2 gene;
a primer for genotyping −989C/G (rs308395) region of FGF2 gene;
a forward primer for amplifying 24684C/G (rs11938826) region of FGF2 gene;
a reverse primer for amplifying 24684C/G (rs11938826) region of FGF2 gene; and
a primer for genotyping 24684C/G (rs11938826) region of FGF2 gene.

5. The test kit for predicting the prognosis of hepatocellular carcinoma according to claim 4, wherein the forward primer for amplifying 16578A/G (rs308428) region of FGF2 gene is a primer of SEQ ID NO. 12;
the reverse primer for amplifying 16578A/G (rs308428) region of FGF2 gene is a primer of SEQ ID NO. 13;
the primer for genotyping 16578A/G (rs308428) region of FGF2 gene is a primer of SEQ ID NO. 33;
the forward primer for amplifying IVS4-81 (DL1002505) region of MTA1 gene is a primer of SEQ ID NO. 21;
the reverse primer for amplifying IVS4-81 (DL1002505) region of MTA1 gene is a primer of SEQ ID NO. 22;
the primer for genotyping IVS4-81 (DL1002505) region of MTA1 gene is a primer of SEQ ID NO. 23;
the forward primer for amplifying −989C/G (rs308395) region of FGF2 gene is a primer of SEQ ID NO. 34;
the reverse primer for amplifying −989C/G (rs308395) region of FGF2 gene is a primer of SEQ ID NO. 35;
the primer for genotyping −989C/G (rs308395) region of FGF2 gene is a primer of SEQ ID NO. 36;
the forward primer for amplifying 24684C/G (rs11938826) region of FGF2 gene is a primer of SEQ ID NO. 37;
the reverse primer for amplifying 24684C/G (rs11938826) region of FGF2 gene is a primer of SEQ ID NO, 38; and
the primer for genotyping 24684C/G (rs11938826) region of FGF2 gene is a primer of SEQ ID NO. 39.

6. A method for predicting prognosis of hepatocellular carcinoma, the method comprising:
a step of obtaining a nucleic acid sample from a clinical specimen; and
a step of determining a nucleotide sequence of at least any one polymorphism regions of polynucleotide selected from the group consisting of GA or AA genotype in IVS4-81 (DL1002505) of MTA1 gene; GG genotype in 24684C/G (rs11938826), GG genotype in −989C/G (rs308395), or GG genotype in 16578A/G (rs308428) of FGF2 gene; and CC or CT genotype in −13021C/T (rs3741208) of IGF2 gene, or a complementary nucleotide thereof.

7. The method for predicting the prognosis of hepatocellular carcinoma according to claim 6, wherein the step of determining the nucleotide sequence of the polymorphism region include a step of hybridizing the nucleic acid sample to a micro-array fixed with the polynucleotide or the complementary nucleotide thereof and a step of detecting a hybridization result thus obtained.

8. A method for screening a drug for improving prognosis of hepatocellular carcinoma, the method comprising:
a step of contacting a polypeptide encoded by the polynucleotide or the complementary nucleotide thereof of the single nucleotide polymorphism (SNP) for predicting prognosis of hepatocellular carcinoma according to claim 1 with a candidate material; and
a step of determining whether the candidate material has activity to enhance or inhibit a function of the polypeptide.

9. The single nucleotide polymorphism (SNP) for predicting the prognosis of hepatocellular carcinoma according to claim 2, wherein the prognosis of hepatocellular carcinoma relates to prognosis of patients treated with curative surgical resection of hepatocellular carcinoma, and is evaluated by using any one marker selected from a rate of tumorigenesis, a rate of recurrence risk, or a survival rate in hepatocellular carcinoma.

10. A method for screening a drug for improving prognosis of hepatocellular carcinoma, the method comprising:
a step of contacting a polypeptide encoded by the polynucleotide or the complementary nucleotide thereof of the single nucleotide polymorphism (SNP) for predicting prognosis of hepatocellular carcinoma according to claim 2 with a candidate material; and
a step of determining whether the candidate material has activity to enhance or inhibit a function of the polypeptide.
